(19) United States
(12) Patent Application Publication
(10) Pub. No.: US 2019/0289806 A1
(43) Pub. Date: Sep. 26, 2019

(54) STEVIA CULTIVARS A01, A03, A05, A06, A07, A08

(71) Applicant: SWEET GREEN FIELDS USA LLC, Bellingham, WA (US)

(72) Inventor: Mel Clinton Jackson, Waikoloa, HI (US)

(21) Appl. No.: 16/358,436

(22) Filed: Mar. 19, 2019

Related U.S. Application Data
(60) Provisional application No. 62/647,036, filed on Mar. 23, 2018.

(51) Int. Cl. A01H 6/14; A01H 5/10

(52) U.S. Cl. CPC A01H 6/1488; A01H 5/10

(57) ABSTRACT

Stevia cultivars, designated A01, A03, A05, A06, A07 and A08, are disclosed. Plant parts of stevia cultivars, the plants of stevia, methods for producing a stevia plant produced by crossing the cultivars with itself or another stevia variety, hybrid stevia seeds and plants produced by crossing the cultivar with another stevia cultivar and plant products e.g. glycosides, are within the scope of the invention.

### CROSS REFERENCE TO RELATED APPLICATIONS

This application claims the benefit of priority under 35 U.S.C. § 119(e) to U.S. Provisional Patent Application No. 62/647,036, filed Mar. 23, 2018. The disclosure set forth in the referenced application is incorporated herein by reference in its entirety.

### BACKGROUND

The present disclosure relates to stevia (Stevia rebaudiana) cultivars designated A01, A03, A05, A06, A07, A08, including seeds, plants, and hybrids. This disclosure further relates to a method for producing stevia seed and plants from A01, A03, A05, A06, A07, A08.

Stevia is an important and valuable field crop for the production of sweeteners, sugar substitutes, and other consumable ingredients. A goal of stevia plant breeders is to develop stable, high yielding stevia cultivars of stevia species that are commercially useful. This means cultivars that have suitable amounts and quality of sweeteners, sugar substitutes, and other consumable ingredients.

The development of new stevia cultivars requires the evaluation and selection of parental plants and crossing of these parents to produce improved cultivars.

### SUMMARY

This disclosure relates to stevia seeds, stevia plants, stevia cultivars, and a method for producing a stevia plant. The cultivars disclosed herein are designated A01, A03, A05, A06, A07 and A08. They are further identified by biological deposits under the Budapest Treaty.

A method of producing stevia seeds and plants includes crossing a plant of one cultivar with a stevia plant of a different genetic cultivar.

Living plant tissue such as shoots, microshoots and seeds of the stevia A01, A03, A05, A06, A07 and A08, plants produced by growing the seed of the stevia A01, A03, A05, A06, A07 and A08, and derivatives of such plants are disclosed.

Also disclosed are tissue cultures of regenerable cells of the stevia cultivars A01, A03, A05, A06, A07, A08, as well as plants regenerated therefrom, wherein the regenerated stevia plant expresses all the physiological and morphological characteristics of a plant grown from the stevia seed or tissue culture designated A01, A03, A05, A06, A07, A08.

A stevia plant of the stevia cultivars A01, A03, A05, A06, A07 or A08 may include at least a first transgene, wherein the stevia plant is otherwise capable of expressing all the physiological and morphological characteristics of the stevia cultivars A01, A03, A05, A06, A07, A08. A plant is provided that includes a single locus conversion. A single locus conversion may comprise a transgenic gene, which has been introduced by genetic transformation into the stevia variety A01, A03, A05, A06, A07, A08 or a progenitor thereof. A transgenic or non-transgenic single locus conversion can also be introduced by backcrossing, as is known in the art. In certain embodiments, the single locus conversion may include a dominant or recessive allele. The locus conversion may confer potentially any desired trait upon the plant as described herein.

Still yet, another embodiment relates to a first generation F₁ hybrid stevia seed produced by crossing a plant of a stevia variety disclosed herein, to a stevia plant of another of the variety disclosed herein, or stevia from a different genetic lineage. That is, the scope of the claims includes F₁ hybrid stevia plants grown from the hybrid seed produced by crossing a stevia A01, A03, A05, A06, A07, A08 to a different stevia plant. Still further included in the invention are the seeds of an F₁ hybrid plant produced with the stevia variety A01, A03, A05, A06, A07 or A08 as one parent, the second generation F₂ hybrid stevia plant grown from the seed of the F₁ hybrid plant, and the seeds of the F₂ hybrid plant.

A method of producing stevia seeds includes crossing a first plant of the stevia variety A01, A03, A05, A06, A07 or A08 to any second stevia plant, including itself or another plant of the variety A01, A03, A05, A06, A07, A08. The method of crossing comprises the steps of: (a) planting seeds of the stevia variety A01, A03, A05, A06, A07, A08; (b) cultivating stevia plants resulting from the seeds until the plants bear flowers; (c) allowing fertilization of the flowers of the plants; and (d) harvesting seeds produced from the plants.

A method of producing hybrid stevia seeds includes crossing a stevia variety A01, A03, A05, A06, A07, A08 to a distinct stevia plant which is non-isogenic to a stevia variety A01, A03, A05, A06, A07, A08. In particular, where the crossing includes: (a) planting seeds of stevia variety A01, A03, A05, A06, A07, A08 and a second, distinct stevia plant; (b) cultivating the stevia plants grown from the seeds until the plants bear flowers; (c) cross pollinating a flower on one of the two plants with the pollen of the other plant; and (d) harvesting the seeds resulting from the cross pollinating.

A method for developing a stevia plant in a stevia breeding program is: (a) obtaining a stevia plant, or its parts, of the variety A01, A03, A05, A06, A07 or A08; and (b) employing the plant or parts as a source of breeding material using plant breeding techniques. In the method, the plant breeding techniques may be selected from the group consisting of recurrent selection, mass selection, bulk selection, backcrossing, pedigree breeding, genetic marker-assisted selection, and genetic transformation. In certain embodiments, the stevia plant of variety A01, A03, A05, A06, A07, A08 is used as the male or the female parent.

A method of producing a stevia plant derived from a stevia variety A01, A03, A05, A06, A07, or A08 includes: (a) preparing a progeny plant derived from a stevia variety A01, A03, A05, A06, A07, A08 by crossing a plant of the stevia variety A01, A03, A05, A06, A07, A08 with a second stevia plant; and (b) crossing the progeny plant with itself or a second plant to produce a progeny plant of a subsequent generation which is derived from a plant of the stevia variety A01, A03, A05, A06, A07, A08. The method optionally includes: (c) crossing the progeny plant of a subsequent generation with itself or a second plant; and (d) repeating steps (b) and (c) for at least 2-10 additional generations to produce an inbred stevia plant derived from the stevia variety A01, A03, A05, A06, A07, A08. Also provided is a plant produced by this and the other methods of the invention. Plant variety A01, A03, A05, A06, A07, A08-derived plants produced by this and the other methods of the invention described herein may, in certain embodiments, be further defined as comprising the traits of plant variety A01, A03, A05, A06, A07, A08 given in Table 1.

A method of vegetatively propagating the stevia plant of the present application, includes: (a) collecting tissue or cells capable of being propagated from a plant of A01, A05, A07, A08; (b) cultivating the tissue or cells of (a) to obtain proliferated shoots; and (c) rooting the proliferated shoots to obtain rooted plantlets; or (d) cultivating the tissue or cells to obtain proliferated shoots, or to obtain plantlets. Further, plants produced by growing the plantlets or proliferated shoots are provided for.

In addition to the exemplary aspects and embodiments described above, further aspects and embodiments may become apparent by study of the following descriptions.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1A-1B is a photograph of the A01 stevia plant; FIG. 1A is a photograph of single leaves; FIG. 1B is a photograph of a growing plant.

FIG. 2A-2B is a photograph of the A03 stevia plant; FIG. 2A is a photograph of single leaves; FIG. 2B is a photograph of a growing plants.

FIG. 3A-3B is a photograph of the A05 stevia plant; FIG. 3A is a photograph of single leaves; FIG. 3B is a photograph of plants with colored sections.

FIG. 4A-4B is a photograph of the A06 stevia plant; FIG. 4A is a photograph of single leaves; FIG. 4B is a photograph of growing plants.

FIG. 5A-5B is a photograph of the A07 stevia plant; FIG. 5A is a photograph of single leaves; FIG. 5B is a photograph of growing plants.

FIG. 6A-6B is a photograph of the A08 stevia plant; FIG. 6A is a photograph of single leaves; FIG. 6B is a photograph of growing plants.

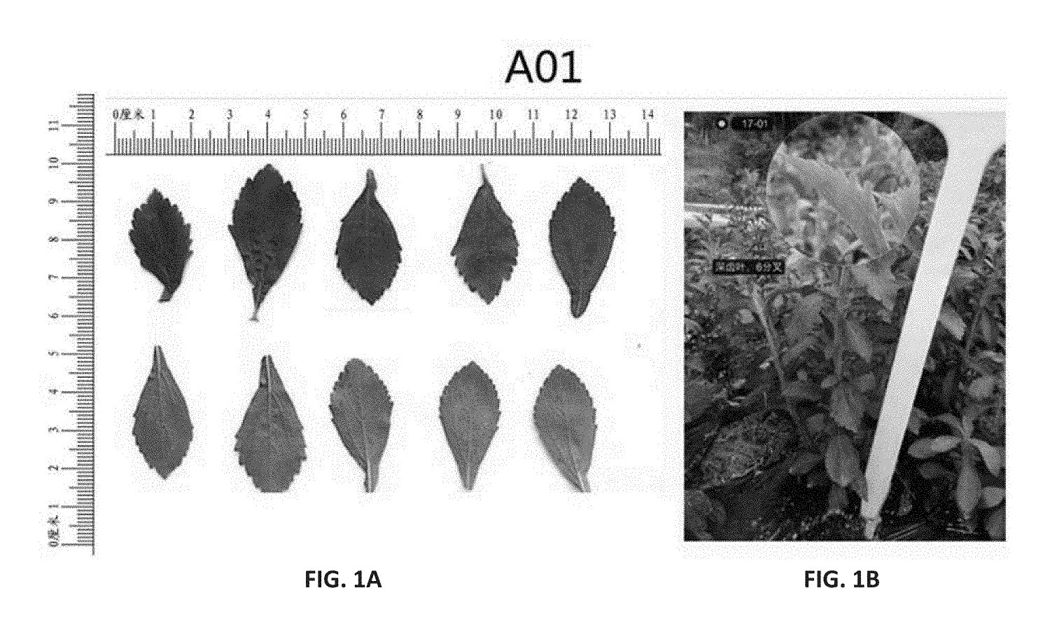

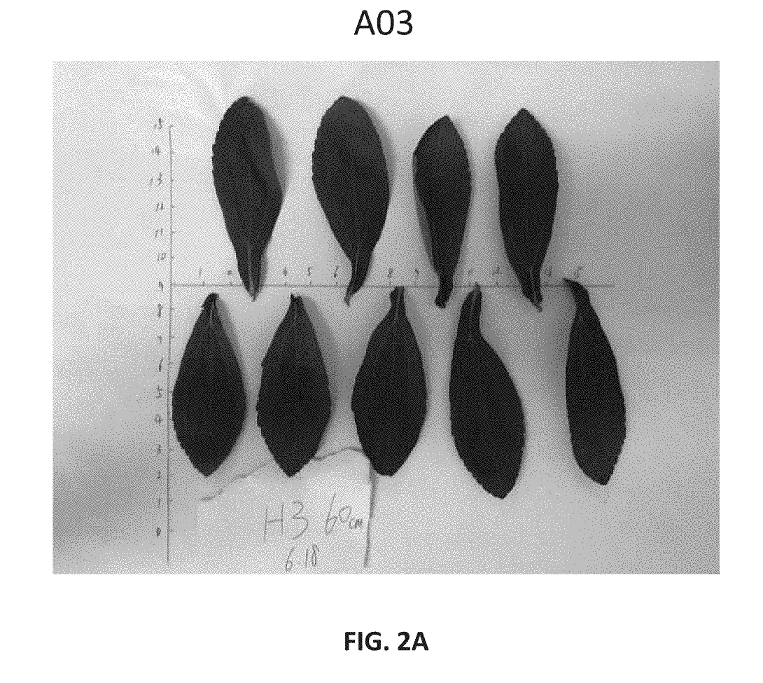

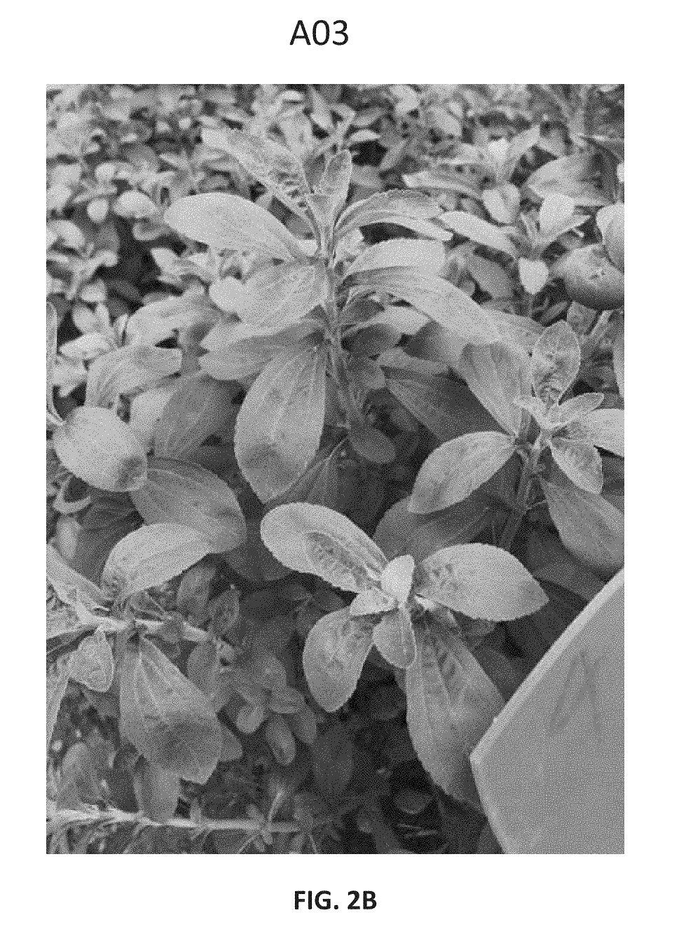

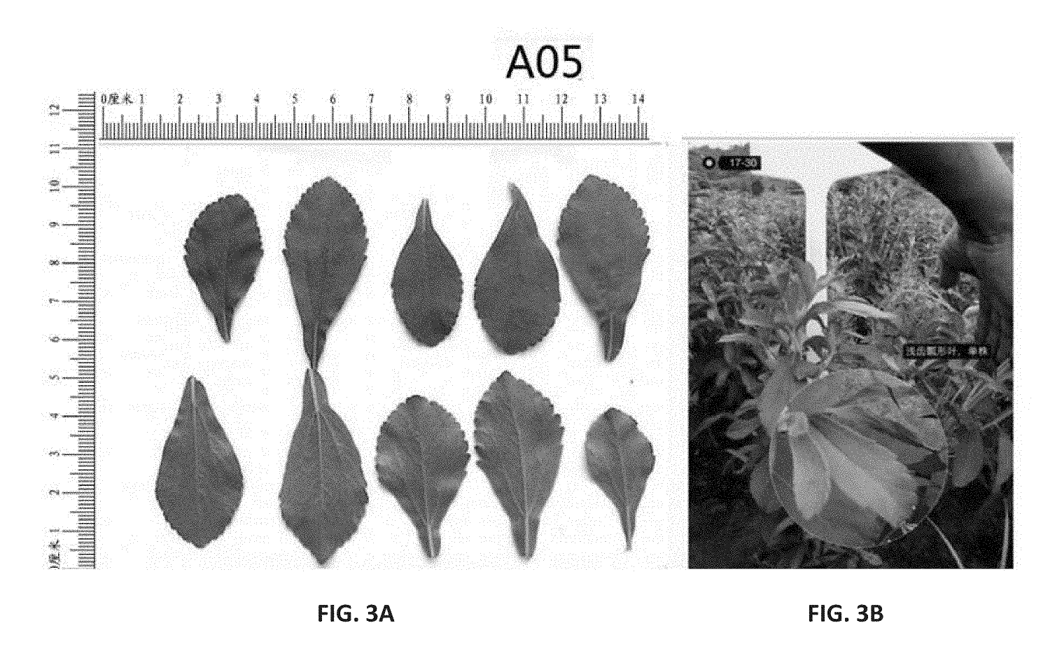

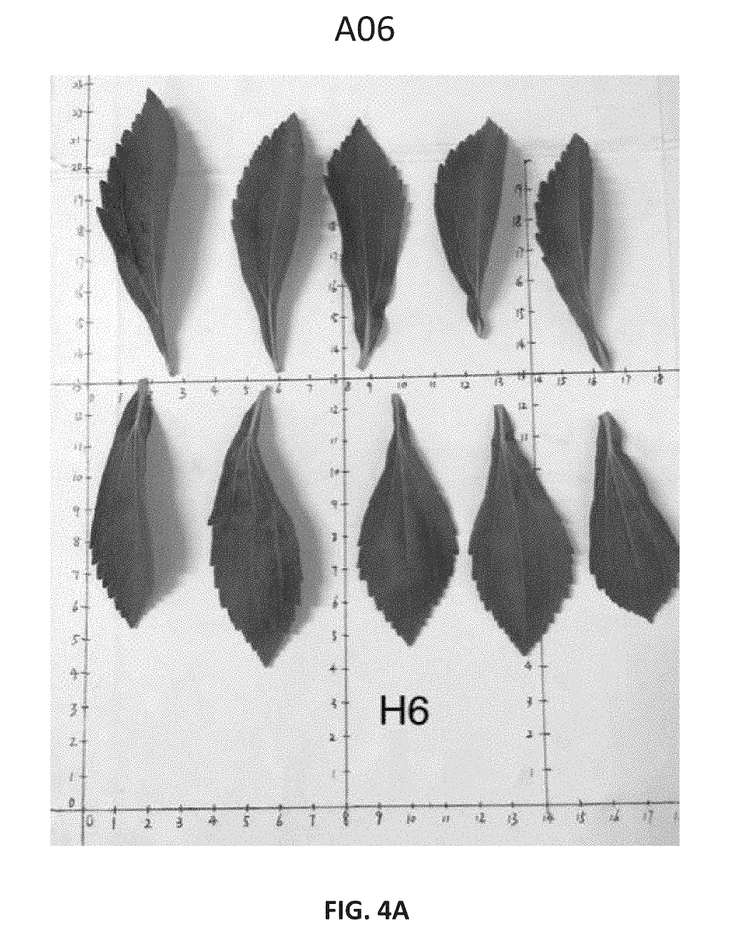

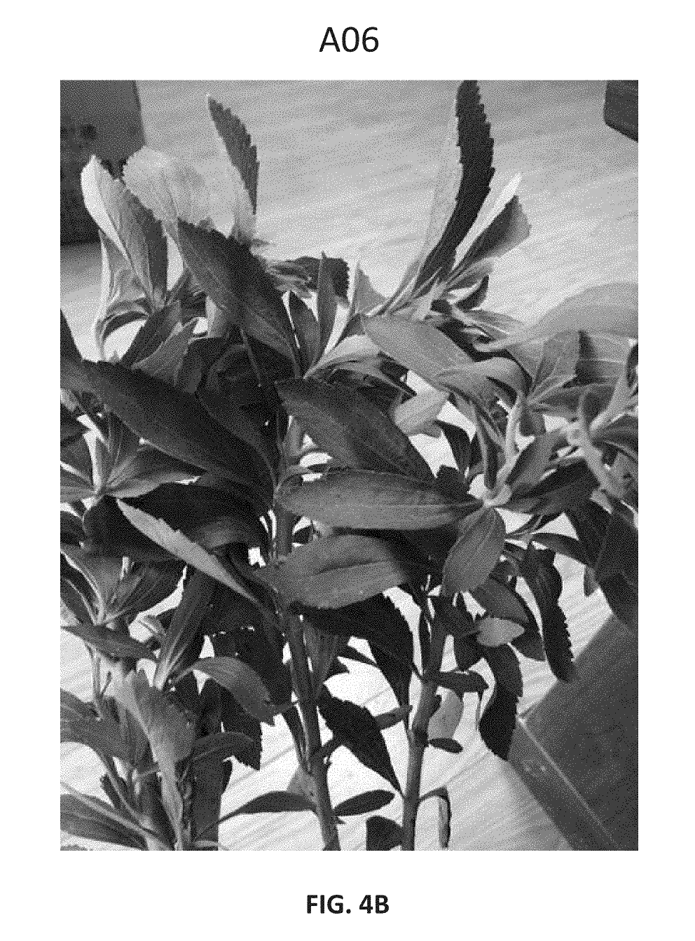

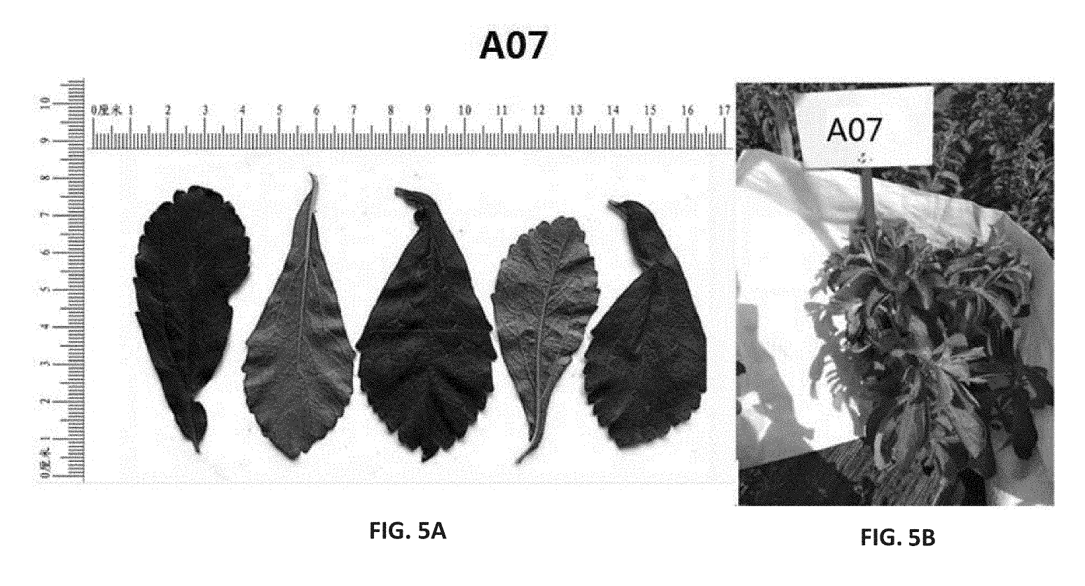

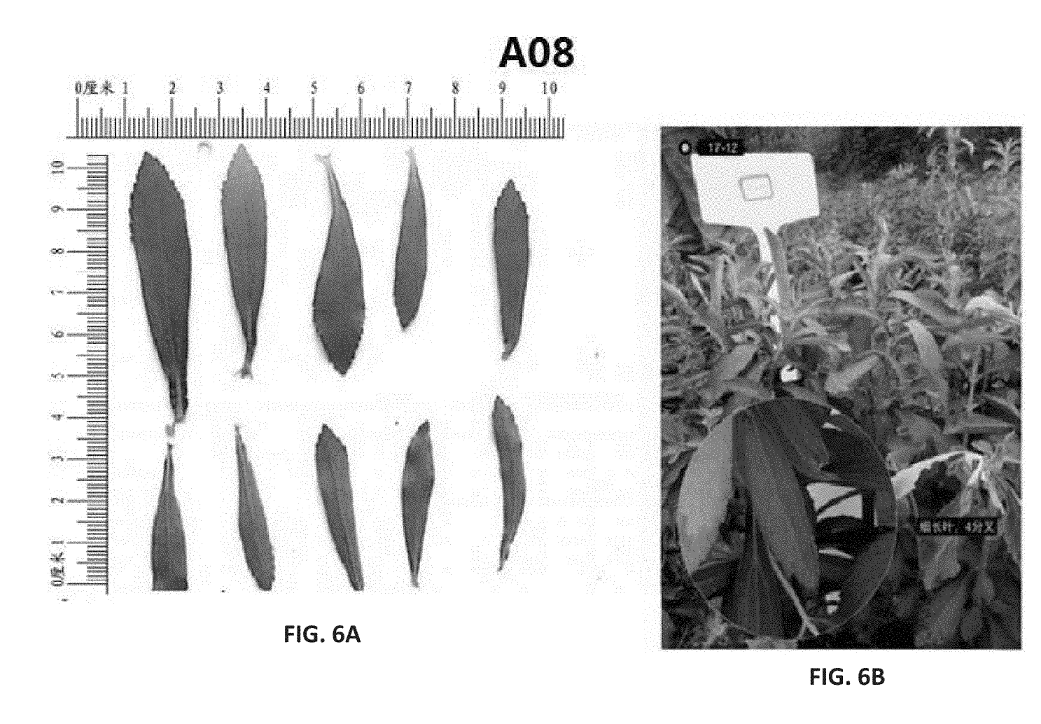

### DETAILED DESCRIPTION

Stevia cultivars A01, A03, A05, A06, A07, A08 are Stevia rebaudiana varieties, which have shown uniformity and stability, as described in Table 1. The varieties have been reproduced a sufficient number of generations with careful attention to uniformity of plant type. The cultivar has been increased with continued observation to uniformity.

Stevia cultivars A01, A03, A05, A06, A07, A08 have the following morphological and physiological characteristics.

TABLE 1
Content of stevia glycosides¹

| | | | | | | | | | | | | | Age |
|---|---|---|---|---|---|---|---|---|---|---|---|---|---|
| No. | RM | RD | RA | TD | RF | RC | DA | RU | RB | St-b | TBG | Moisture | (Days) |
| A01 | 0.82 | 1.36 | 14.32 | 1.52 | 0.33 | 0.69 | 1.19 | 0.08 | 0.13 | 0.07 | 19.69 | 9.5 | 85 |
| A05 | NS | 1.68 | 13.99 | 2.51 | 0.26 | 1.9 | 0.21 | 0.11 | 0.13 | NS | 20.79 | 14.3 | 56 |
| A07 | NS | 0.84 | 3.67 | 12.49 | 0.17 | 1.41 | 1.25 | 0.00 | 0.05 | 0.06 | 19.92 | 17.5 | 40 |
| A08 | 0.66 | 2.4 | 16.45 | 1.7 | 0.43 | 0.76 | 1.25 | 0.06 | 0.08 | 0.1 | 23.22 | 8.5 | 78 |

¹Age is days after planting;
TD = Stevioside;
St-b = Steviol bioside;
TBG = Total stevial glycosides.

Description: All cultivars are Stevia rebaudiana L. Bertoni, geographical source: China.

A01: Appearance: Half-circle shape of leaves, there are visible deep serration-like lines from top to middle of leaves;

Character of stevia glycosides: High Reb A (RA) and total stevia glycosides, highest content shows up around growth of 80˜90 days.

A03: Leaves are oval shaped, thick, distance of leaves is short, stem is strong, RA content is in range of 9.01˜11.31%.

A05: Appearance: Long leaves, there is visible serration-like lines from top to middle of leaves.

A06: Leaves are spindle form, with a sawtooth edge, thick leaves, distance between leaves is short, stem is strong, RA content: 9.41˜12.22%, total stevia glycosides: 14.37˜17.92%.

Character of stevia glycosides: High Reb A content and total glycosides, highest content shows up around 50˜70 days.

A07: Appearance: Big leaves, there is visible deep serration-like lines on top of leaves.

Character of stevia glycosides: High stevioside content and total stevia glycosides content, highest content shows up around 40˜50 days.

A08 Appearance: Thin and long leaves, there are few serration-like lines on top of leaves.

Character of stevia glycosides: High Reb A and total stevia glycosides content, highest content shows up around 70˜80 days.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to popular cultivars in environments representative of the commercial target area(s) for three or more years. The lines having superiority over the popular cultivars are candidates to become new commercial cultivars. Those lines still deficient in a few traits are discarded or utilized as parents to produce new populations for further selection.

These processes, which lead to the final step of marketing and distribution, usually take from seven to twelve years from the time the first cross is made. Therefore, development of new cultivars is a time-consuming process that requires precise forward planning, efficient use of resources, and a minimum of changes in direction.

A most difficult task is the identification of individuals that are genetically superior because for most traits the true genotypic value is masked by other confounding plant traits or environmental factors. One method of identifying a superior plant is to observe its performance relative to other experimental lines and widely grown standard cultivars. For many traits a single observation is inconclusive, and replicated observations over time and space are required to provide a good estimate of a line's genetic worth.

Each year, the plant breeder selects the germplasm to advance to the next generation. This germplasm is grown under unique and different geographical, climatic, and soil conditions and further selections are then made, during and at the end of the growing season. The lines which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce, with any reasonable likelihood, the same cultivar twice by using the exact same original parents and the same selection techniques. This unpredictability results in the expenditure of large amounts of research moneys to develop superior new stevia cultivars.

Pureline cultivars of stevia are commonly bred by hybridization of two or more parents followed by selection. The complexity of inheritance, the breeding objectives, and the available resources influence the breeding method. Pedigree breeding, recurrent selection breeding, and backcross breeding are breeding methods commonly used in self-pollinated crops such as stevia. These methods refer to the manner in which breeding pools or populations are made in order to combine desirable traits from two or more cultivars or various broad-based sources. The procedures commonly used for selection of desirable individuals or populations of individuals are called mass selection, plant-to-row selection, and single seed descent or modified single seed descent. One or a combination of these selection methods can be used in the development of a cultivar from a breeding population.

Pedigree breeding is primarily used to combine favorable genes into a totally new cultivar that is different in many traits than either parent used in the original cross. It is commonly used for the improvement of self-pollinating crops. Two parents which possess favorable, complementary traits are crossed to produce an F₁ (filial generation 1). An F₂ population is produced by selfing F₂ plants. Selection of desirable individual plants may begin as early as the F₂ generation wherein maximum gene segregation occurs. Individual plant selection can occur for one or more generations. Successively, seed from each selected plant can be planted in individual, identified rows or hills, known as progeny rows or progeny hills, to evaluate the line and to increase the seed quantity, or to further select individual plants. After a progeny row or progeny hill is selected as having desirable traits, it becomes what is known as a breeding line that is specifically identifiable from other breeding lines that were derived from the same original population. At an advanced generation (i.e., F₅ or higher) seed of individual lines are evaluated in replicated testing. At an advanced stage the best lines or a mixture of phenotypically similar lines from the same original cross are tested for potential release as new cultivars.

The single seed descent procedure in the strict sense refers to planting a segregating population, harvesting one seed from every plant, and combining these seeds into a bulk, which is planted as the next generation. When the population has been advanced to the desired level of inbreeding, the plants from which lines are derived will each trace to different F₂ individuals. Primary advantages of the seed descent procedures are to delay selection until a high level of homozygosity (e.g., lack of gene segregation) is achieved in individual plants, and to move through these early generations quickly, usually through using winter nurseries.

The modified single seed descent procedures involve harvesting multiple seed (i.e., a single lock or a simple boll) from each plant in a population and combining them to form a bulk. Part of the bulk is used to plant the next generation and part is put in reserve. This procedure has been used to save labor at harvest and to maintain adequate seed quantities of the population.

Selection for desirable traits can occur at any segregating generation (F.sub.2 and above). Selection pressure is exerted on a population by growing the population in an environment where the desired trait is maximally expressed and the individuals or lines possessing the trait can be identified. For instance, selection can occur for disease resistance when the plants or lines are grown in natural or artificially-induced disease environments, and the breeder selects only those individuals having little or no disease and are thus assumed to be resistant.

In addition to phenotypic observations, the genotype of a plant can also be examined. There are many laboratory-based techniques available for the analysis, comparison, and characterization of plant genotype. Among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs—which are also referred to as Microsatellites), and Single Nucleotide Polymorphisms (SNPs).

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The use of molecular markers in the selection process is often called Genetic Marker Enhanced Selection. Molecular markers may also be used to identify and exclude certain sources of germplasm as parental varieties or ancestors of a plant by providing a means of tracking genetic profiles through crosses as discussed more fully hereinafter.

Mutation Breeding

Mutation breeding is another method of introducing new traits into stevia varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogues like 5-bromo-uracil), antibiotics, alkylating agents (such as sulfur mustards, nitrogen mustards, epoxides, ethylenamines, sulfates, sulfonates, sulfones, or lactones), azide, hydroxylamine, nitrous acid, or acridines. After a desired trait is observed through mutagenesis the trait may then be incorporated into existing germplasm by traditional breeding techniques.

Production of Double Haploids

The production of double haploids can also be used for the development of homozygous varieties in a breeding program. Double haploids are produced by the doubling of a set of chromosomes from a heterozygous plant to produce a completely homozygous individual.

The stevia flower is monoecious in that the male and female structures are in the same flower. The crossed or hybrid seed is produced by manual crosses between selected parents. Floral buds of the parent that is to be the female are emasculated prior to the opening of the flower by manual removal of the male anthers. At flowering, the pollen from flowers of the parent plants designated as male, are manually placed on the stigma of the previous emasculated flower. Seed developed from the cross is known as first generation F₁ hybrid seed. Planting of this seed produces F₁ hybrid plants of which half their genetic component is from the female parent and half from the male parent. Segregation of genes begins at meiosis thus producing second generation F₂ seed. Assuming multiple genetic differences between the original parents, each F₂ seed has a unique combination of genes.

Other objects, features, and advantages may become apparent from the following detailed description. It should be understood, however, that the detailed description and the specific examples, while indicating specific embodiments, are given by way of illustration only, since various changes and modifications within the spirit and scope of the embodiments of the invention may become apparent to those skilled in the art from this detailed description.

### Definitions

The following definitions are used in the specification and claims.

Cultivar: Cultivated variety.

Genotype: Refers to the genetic composition of a cell or organism.

Plant: As used herein, the term “plant” includes reference to an immature or mature whole plant, including a plant that has been processed for steviol glycosides. Seed or plant parts that will produce the plant is also considered to be the plant; includes plant cells, plant protoplasts, plant cell tissue cultures from which stevia plants can be regenerated, plant calli, plant clumps, and plant cells that are intact in plants or parts of plants, such as embryos, pollen, ovules, flowers, leaves, roots, root tips, anthers, and pistils.

Plant Part: As used herein, the term “plant part” includes leaves, stems, roots, root tips, seed, embryo, pollen, ovules, flowers, root tips, shoots, microshoots, anthers, tissue, and cells.

Rebaudioside A: As used herein is a steviol glycoside that contains only glucose as its monosaccharide moieties. It contains four glucose molecules in total with the central glucose of the triplet connected to the main steviol structure at its hydroxyl group, and the remaining glucose at its carboxyl group forming an ester bond.

Rebaudioside D: As used herein is an ent-kaurane diterpene glycoside isolated from Stevia rebaudiana.

Rebaudioside M: As used herein is an ent-kaurane diterpene glycoside isolated from Stevia rebaudiana.

SNP: As used herein, the term “SNP” shall refer to a single nucleotide polymorphism.

Stevioside content: As used herein, stevioside is the percent glycoside derived from the stevia plant.

Traditional breeding techniques: Encompasses herein crossing, selfing, selection, double haploid production, embryo rescue, protoplast fusion, marker assisted selection, mutation breeding etc. as known to the breeder (i.e. methods other than genetic modification/transformation/transgenic methods), by which, for example, a genetically heritable trait can be transferred from one carrot line or variety to another.

Vegetative propagation: “Vegetative reproduction” or “clonal propagation” are terms used interchangeably herein and mean the method of taking part of a plant and allowing that plant part to form at least roots where plant part is, e.g., defined as or derived from (e.g. by cutting of) leaf, pollen, embryo, cotyledon, hypocotyl, cells, nodes, protoplasts, meristematic cell, root, root tip, pistil, anther, flower, shoot tip, shoot, stem, petiole, etc. When a whole plant is regenerated by vegetative propagation, it is also referred to as a vegetative propagation.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, leaves, stems, roots, root tips, anthers, and pistils. Means for preparing and maintaining plant tissue culture are well known in the art. By way of example, a tissue culture comprising organs has been used to produce regenerated plants.

The use of the word “a” or “an” when used in conjunction with the term “comprising” in the claims and/or the specification may mean “one,” but it is also consistent with the meaning of “one or more,” “at least one,” and “one or more than one.”

### DEPOSITS UNDER THE BUDAPEST TREATY

Tissue culture (callus) samples from each of the 6 stevia varieties have been deposited by the inventor on behalf of the Assignee, Sweet Green Fields USA, LLC, in the American Type Culture Collection (ATCC), 10801 University Boulevard, Manassas, Va. 20110, United States of America on March ______, 2019. PTA accession numbers are A01-PTA ______, A03-PTA ______, A05-PTA ______, A06-PTA ______, A07-PTA ______, A08-PTA ______. All restrictions will be removed upon granting of a patent, and the deposits are intended to meet all of the requirements of 37 C.F.R. §§ 1.801-1.809, and satisfy the Budapest Treaty requirements. The deposit will be maintained in the depository for a period of thirty years, or five years after the last request, or for the enforceable life of the patent, whichever is longer, and will be replaced as necessary during that period.

### PUBLICATIONS

These publications are incorporated by reference to the extent they relate materials and methods disclosed herein.

U.S. Pat. No. 9,668,450 B2

WO 2016/034942 A1

1. A plant of a Stevia rebaudiana cultivar selected from the group consisting of cultivars designated A01, A03, A05, A06, A07 and A08, wherein representative samples of live plant tissue of the cultivars were deposited in the ATCC depository and provided accession numbers PTA-______, ______, ______, ______, ______, ______.

2. A plant, or a plant part thereof consisting of leaves, cotyledons, hypocotyl, meristematic cells, ovules, seeds, cells, roots, root tips, pistils, anthers, flowers, and stems, produced by growing the plant of claim 1.

3. A stevia plant, or part thereof, having all of the physiological and morphological characteristics of the stevia plant of claim 1.

4. An extract of the stevia plant obtained from the plant of claim 1.

5. The extract of claim 4 is a glycoside.

6. A method of producing a food or feed product, the method comprising obtaining the plant or plant part of claim 2, and producing the product.

7. A tissue or cell culture of regenerable cells produced from the plant of claim 1.

8. The tissue or cell culture of claim 7, comprising tissues or cells from a plant part selected from the group consisting of leaves, cotyledons, hypocotyl, meristematic cells, roots, root tips, pistils, anthers, flowers, and stems.

9. A stevia plant regenerated from the tissue or cell culture of claim 8, wherein the plant has all of the morphological and physiological characteristics of stevia cultivars listed in Table 1.

10. A method of vegetatively propagating a stevia plant, the method comprising:
a. collecting tissue or cells capable of being propagated from the plant according to claim 1;
b. cultivating the tissue or cells of (a) to obtain proliferated shoots; and
c. rooting said the shoots to obtain rooted plantlets; or
d. cultivating said tissue or cells to obtain proliferated shoots, or plantlets.

11. A stevia plant produced by growing the plantlets or proliferated shoots of claim 10.

12. A method for producing an F₁ stevia seed, wherein the method comprises crossing the plant of claim 1 with a different stevia plant and harvesting the resultant F₁ stevia seed.

13. A stevia seed produced by the method of claim 12.

14. A stevia plant, or a part thereof, produced by growing the seed of claim 13.

15. A method of producing a herbicide resistant stevia plant, wherein the method comprises transforming the stevia plant of claim 1 with a transgene, wherein the transgene confers resistance to a herbicide chosen from glyphosate, sulfonylurea, imidazolinone, dicamba, glufosinate, phenoxy proprionic acid, cyclohexanedione, L-phosphinothricin, triazine, benzonitrile, and bromoxynil.

16. A herbicide resistant stevia plant produced by the method of claim 15.

17. A method of producing an insect resistant stevia plant, wherein the method comprises transforming the stevia plant of claim 1 with a transgene that confers insect resistance.

18. An insect resistant stevia plant produced by the method of claim 17.

19. The stevia plant of claim 18, wherein the transgene encodes a Bacillus thuringiensis endotoxin.

20. A method of producing a disease resistant stevia plant, wherein the method comprises transforming the stevia plant of claim 1 with a transgene that confers disease resistance.

21. A disease resistant stevia plant produced by the method of claim 20.

22. A method for developing a stevia plant in a stevia plant breeding program, comprising applying plant breeding techniques selected from the group consisting of a marker enhanced selection, haploid/double haploid production, and transformation, to the stevia plant of claim 1, or its parts, wherein the application of said techniques results in development of a stevia plant.

23. A Stevia rebaudiana seed, plant, plant part, or cell produced by crossing a plant or plant part of Stevia rebaudiana cultivar A01, A02, A03, A05, A06, A07 and A08, or a locus conversion thereof, with another Stevia rebaudiana plant.

24. A plant part consisting of pollen or embryos produced by growing the plant of claim 1.
